(19) United States
(12) Patent Application Publication
(10) Pub. No.: US 2020/0108209 A1
(43) Pub. Date: Apr. 9, 2020

(54) NEEDLE EJECTION AND RETRACTION MECHANISM AND INJECTOR DEVICE

(71) Applicant: Novartis AG, Basel (CH)

(72) Inventors: Alain Schmidlin, Basel (CH); Mario Iobbi, Basel (CH); Erich Studer, Basel (CH); Andrew Bryant, Basel (CH); Chinmay Deodhar, Pune (IN); Rajan Patel, Menlo Park, CA (US)

(21) Appl. No.: 16/494,236

(22) PCT Filed: Feb. 1, 2018
(86) PCT No.: PCT/EP2018/052556

§ 371 Date: Sep. 13, 2019

Related U.S. Application Data
(60) Provisional application No. 62/471,992, filed on Mar. 16, 2017.

(30) Foreign Application Priority Data
Mar. 31, 2017 (EP) 17164081.6

(51) Int. Cl. A61M 5/32; A61M 5/20

(52) U.S. Cl. CPC A61M 5/3232; A61M 5/20; A61M 2005/31518; A61M 2005/206; A61M 5/3234

(57) ABSTRACT

A needle ejection and retraction mechanism (10) comprises a needle (12), a needle hub (14) connected to the needle (12) and adapted to be displaced in a distal direction and in a proximal direction, a first spring mechanism (16) compressed to exert a force in the distal direction on the needle hub (14) and adapted to cause the needle (12) to eject in the distal direction, a second spring mechanism (18) compressed to exert a force in the proximal direction on the needle hub (14) and adapted to cause the needle (12) to retract in the proximal direction after it has been ejected in the distal direction, and an actuator (20) which is adapted to activate the exertion of the force in the distal direction on the needle hub (14) and the exertion of the force in the proximal direction on the needle hub (14). An injector device comprises a needle ejection and retraction mechanism (10), a control unit which is adapted to control the actuator (20), and a sensor unit which is connected to a control unit and which is adapted to determine, after the needle (12) has at least been partially ejected from the injector device, a value which indicates a distance between the sensor unit and the skin of a human or animal body, wherein the control unit is adapted, upon determination that the value exceeds a predetermined threshold value, to cause the actuator to activate the exertion of the force in the proximal direction on the needle hub (14) so that a distal tip (13) of the needle (12) does not protrude from the injector device.

The invention relates to a needle ejection and retraction mechanism, in particular, a needle ejection and retraction mechanism that can be used in an injector device. Furthermore, the invention relates to an injector device comprising a needle ejection and retraction mechanism.

Needles may be injected into the skin of a human or animal body for various reasons, for example, in order to deliver a drug into the human or animal body or to puncture the human or animal body for a biopsy examination. For this, various spring-based, motor-based and gas-based mechanisms are known, which cause a needle to be ejected out of a housing and to be injected into the skin of the human or animal body.

Since the ejected injection needle is a source of injury for the user, mechanisms are known which retract the injection needle back into the housing after the injection process has been completed.

U.S. Pat. No. 7,749,194 B2 concerns an auto-injector comprising a vial storing an injectable medicament. The auto-injector further comprises a gas container, an injection needle and a retraction spring. Upon escape of the gas contained in the gas container, the injection needle is ejected from the housing of the auto-injector thereby compressing the retraction springs. After the injection of the medicament via the needle into the skin of a human body, the retraction spring causes the needle to be retracted into the housing.

GB 143 084 A concerns an injection device of the type that receives a syringe, extends it, discharges its contents and then retracts it automatically.

WO 2014/194183 A2 concerns a fluid delivery device comprising a housing having a bottom surface configured to be coupled to a skin surface. The fluid delivery device includes a cartridge prefilled with a fluid and configured to be inserted into the housing. The cartridge has a septum configured to be generally perpendicular to the bottom surface when the cartridge is inserted in the housing. The fluid delivery device includes a needle assembly that has a needle that includes a fluid coupling end and a delivery end. The fluid coupling end of the needle is fluidly disengaged from the cartridge in an initial position. The delivery end of the needle extends past the plane of the bottom surface and the fluid coupling end of the needle extends through the septum in a deployed position.

The invention is directed at the object of providing a needle ejection and retraction mechanism which allows an instantaneous and swift needle ejection and retraction. Furthermore, the invention is directed at the object of providing an injector device which allows an instantaneous and swift needle ejection and retraction.

This object is addressed by a needle ejection and retraction mechanism as defined in claim 1, and an injector device as defined in claim 14.

The needle ejection and retraction mechanism comprises a needle, a needle hub connected to the needle and adapted to be displaced in a distal direction and in a proximal direction, a first spring mechanism compressed to exert a force in the distal direction on the needle hub and adapted to cause the needle to eject in the distal direction, a second spring mechanism compressed to exert a force in the proximal direction on the needle hub and adapted to cause the needle to retract in the proximal direction after it has been ejected in the distal direction, and an actuator which is adapted to activate the exertion of the force in the distal direction on the needle hub and the exertion of the force in the proximal direction on the needle hub.

The distal direction in the sense of the present disclosure is a direction in which the needle can be extracted from its initial position. Accordingly, the proximal direction in the sense of the present disclosure is a direction opposite, or substantially opposite, to the distal direction, i.e., a direction in which the needle can be retracted after the extraction.

The needle may be any kind of needle which is adapted to be injected into the skin of a human or animal body, for example, a hollow needle having a needle tip at its distal end, a puncturing needle, a biopsy needle, or a drainage needle. The needle hub may be any kind of protrusion, for example a flange, which is connected or integrally provided with the needle and allows a displacement of the needle hub together with the needle in the distal direction and in the proximal direction.

Each of the first and second spring mechanisms may comprise any kind of spring that is adapted to store and release mechanical energy, for example, a coil spring made of a steel alloy. The first and second spring mechanisms are not limited to single springs but may each be realized by a plurality of springs. Moreover, the first and the second spring mechanisms may be adapted to directly or indirectly, e.g., via another element, contact the needle hub.

Preferably, the first and second spring mechanisms are disposed such in the needle ejection and rejection mechanism that they are biased in opposite directions. Thus, the first spring mechanism is adapted to exert a force in the distal direction, whereas the second spring mechanism is adapted to exert a force in the proximal direction. Since a spring-based system for both the needle ejection and retraction is foreseen, a swift needle ejection and retraction can be provided.

The actuator may be any kind of actuation means that is adapted to convert a supplied energy, e.g., electric current, hydraulic fluid pressure, or pneumatic pressure, into mechanical motion in order to exert a force. Preferably, the actuator, which is adapted to activate the exertion of the force in the distal direction on the needle hub and the exertion of the force in the proximal direction on the needle hub, is a motor. Since an actuator is used for the activation of the exertion and retraction forces, an instantaneous and precise control of the needle ejection and retraction process can be provided. Furthermore, since only a single actuator is foreseen, a compact and small-sized needle ejection and retraction mechanism can be obtained.

In order to provide a further advanced needle ejection and retraction mechanism, the second spring mechanism may be adapted to exert a higher force than the first spring mechanism. Particularly, the second spring mechanism may be adapted to exert a higher initial and final spring force than the first spring mechanism. Moreover, the second spring mechanism may have a higher spring constant than the first spring mechanism.

The second spring mechanism is adapted to cause a compression of the first spring mechanism after the needle has been ejected in the distal direction. In particular, after the exertion of the force in the distal direction on the needle hub and the ejection of the needle in the distal direction, i.e., after the first spring mechanism is completely or partially released, the second spring mechanism may still be compressed in its initial state. Due to the second spring mechanism causing a higher force than the first spring mechanism, after the exertion of the force in the proximal direction on the needle hub by the second spring mechanism, the first spring mechanism is compressed again. For example, the needle hub may be pushed by the second spring mechanism in the proximal direction and thereby compress the first spring mechanism again. Thus, a swift needle ejection and retraction is provided.

Moreover, the needle retraction can be started even in case the needle is not completely ejected.

In a preferred embodiment, the needle ejection and retraction mechanism comprises a first spring force activation mechanism and a second spring force activation mechanism. The first spring force activation mechanism is adapted to hold the first spring mechanism in the compressed state and to release the first spring mechanism from the compressed state in order to exert the force in the distal direction on the needle hub. The second spring force activation mechanism is adapted to hold the second spring mechanism in the compressed state and release the second spring mechanism from the compressed state in order to exert the force in the proximal direction on the needle hub. For example, the actuator may be coupled, e.g., via one or more gears and further elements, to both the first and the second spring force activation mechanism, and may selectively cause the first and the second spring mechanism from the compressed state in order to exert the force in the proximal direction on the needle hub. For example, the actuator may be coupled, e.g., via one or more gears and further elements, to both the first and the second spring force activation mechanism, and may selectively cause the first and the second spring activation mechanisms to release the first and the second spring mechanisms in order to exert their forces, which causes a selective ejection and retraction of the needle. Thus, a precise and fine-controllable mechanism for needle ejection and retraction is provided.

Preferably, the first spring force activation mechanism comprises a first lever which is adapted to hold the first spring mechanism in the compressed state, and the second spring force activation mechanism comprises a second lever which is adapted to hold the second spring mechanism in the compressed state. The first lever and the second lever may be realized as any kind of holding means that are adapted to hold the first and the second spring mechanisms in the compressed states, and selectively release the first and second spring mechanisms from their compressed states.

In order to provide a compact and space-saving structure, at least one of the first lever and the second lever may be adapted to be moved in a direction that differs from the distal direction and the proximal direction, i.e., differs by a predetermined angle from the direction in which the needle is ejected and retracted. In a preferred embodiment, at least one of the first lever and the second lever is adapted to be moved in a direction that is perpendicular to the distal direction and the proximal direction. Thus, a flat needle ejection and retraction mechanism can be provided. For example, in case the needle ejection and retraction mechanism is employed in an injector device, the injector device may have a flat shape, for example, similar to the shape of a computer mouse. Because of its flat structure, the injector device may be provided with a relatively large skin contact surface which facilitates a stable placement of the injector device on the skin of a human or animal body and a safe injection of the needle into the skin of a human or animal body.

Preferably, the first lever is adapted to be rotated around an axis that is substantially parallel to the distal direction and the proximal direction, i.e., the direction in which the needle is ejected and retracted, after it has been moved in a direction that differs from the distal direction and the proximal direction. The term “substantially parallel” comprises a range from +45 degrees to −45 degrees around an axis that is parallel to the distal direction and the proximal direction. In a preferred embodiment, the first lever is rotated by 90 degrees.

The needle ejection and retraction mechanism may further comprise a ratchet mechanism, which is coupled to the actuator, the first spring force activation mechanism and the second spring force activation mechanism. The ratchet mechanism may be any kind of mechanical device that allows continuous linear or rotary motion in only one direction while preventing motion in the opposite direction. The coupling of the ratchet mechanism to the actuator, the first spring activation mechanism, and the second spring force activation mechanism may, for example, be realized by means of one or a plurality of gears. Preferably, the ratchet mechanism is adapted to activate the exertion of the force in the distal direction on the needle hub and thereafter activate the exertion of the force in the proximal direction on the needle hub. Accordingly, the ratchet mechanism is adapted to act as an interface between the actuator and the first and second spring force activation mechanisms.

In a preferred embodiment, the ratchet mechanism comprises a ratchet and a pawl, wherein the ratchet comprises a plurality of teeth. Preferably, the plurality of teeth is realized as a toothed rack. Each tooth may be uniform but asymmetrical, with each tooth having a moderate slope on one edge and a steeper or vertical slope on the other edge. Alternatively, the teeth may have a saw-shaped profile. Preferably, the pawl is adapted to run in one direction over the plurality of teeth and engage with one of the plurality of teeth when being moved in the opposite direction. The ratchet may have any kind of shape, for example, a linear or a round shape, which allows a running of the pawl over the teeth and an engagement of the pawl with one of the plurality of teeth. Thus, when the pawl moves in the unrestricted (i.e., forward) direction, the pawl slides up and over the edges of the teeth, for example, with a spring forcing it into the depression between the teeth as it passes the tip of each tooth. When the pawl moves in the opposite (backward) direction, the pawl catches against the edge of the first tooth it encounters, thereby locking it against the tooth and moving the ratchet in the opposite (backward) direction.

Further preferably, the pawl is coupled to the first spring force activation mechanism, and the ratchet is coupled to the second spring force activation mechanism. Both couplings may be a direct coupling, or a coupling via one or a plurality of gears.

In a preferred embodiment, the ratchet comprises a surface which faces away from the plurality of teeth and an opening which extends along the plurality of teeth and is provided between the plurality of teeth and the surface that faces away from the plurality of teeth. Thus, when the pawl is moved over the plurality of teeth in the direction away from the first spring mechanism, the part of the ratchet comprising the plurality of teeth flexes towards the surface which faces away from the plurality of teeth so that the pawl can slide over the edges of the teeth without having to move up and down. Thus, a spring forcing the pawl into the depression between the teeth as it passes the tip of each tooth can be avoided, which leads to a more compact structure.

Further preferably, a surface of the ratchet which is facing the pawl comprises a first area and a second area, wherein the second area is closer to the first and second spring mechanisms than the first area, the first area comprises the plurality of teeth, the second area comprises no teeth and extends along the opening that is provided in the ratchet, and the pawl is adapted to be moved along the second area and the first area. Thus, during operation of the ratchet mechanism, it can be ensured that the pawl can slide over the edge of the first tooth of the plurality of teeth, i.e., the tooth being closest to the first spring mechanism.

To activate the exertion of the force in the distal direction on the needle hub, the actuator may be adapted to move the pawl in a first direction away from the first spring mechanism. This movement of the pawl in the first direction may cause the first lever to be moved in a direction that differs from the direction in which the needle is ejected and retracted. For this, the actuator may, for example, be coupled via a gear to the pawl. Upon exertion of the force on the gear, the pawl may be moved in a first direction away from the first spring mechanism, which causes the pawl to run over the plurality of teeth of the ratchet. Additionally the pawl may be coupled to the first lever such that the movement of the pawl in the first direction causes the movement of first lever in the direction that differs from the direction in which the needle is ejected and retracted, thereby releasing the first spring mechanism.

To activate the exertion of the force in the proximal direction on the needle hub, the actuator may further be adapted to move the pawl in a second direction towards the first spring mechanism. Preferably, this movement is provided after the actuator has lo moved the pawl in the first direction. Additionally the pawl may be coupled to the second lever such that the movement of the pawl in the second direction causes a movement of the second lever in a direction that differs from the direction in which the needle is ejected and retracted, thereby releasing the second spring mechanism.

To activate the exertion of the force in the proximal direction on the needle hub, the pawl may be adapted, when being engaged with one of the plurality of teeth of the ratchet, to move the ratchet in the second direction towards the first spring mechanism. Thus, when the pawl is engaged with anyone of the plurality of the teeth, it is possible to instantaneously move the ratchet in the second direction.

In a preferred embodiment, in order to force a drug out of a drug storage cartridge, the needle ejection and retraction mechanism comprises a push mechanism which is coupled to at least one of the pawl and the first spring force activation mechanism. Upon movement of the pawl over and along the plurality of the teeth, the push mechanism forces a drug out of the drug storage cartridge. Thus, moving of the pawl and the first spring force activation mechanism in the first direction away from the first and second spring mechanisms has a double function of initiating the extraction of the needle and of initiating a drug delivery process.

In order to enable a space-saving design, the push mechanism may comprise a spring guide and slinky spring which is adapted to be moved in the spring guide, wherein the spring guide has a curved shape. For example, the spring guide may be substantially U-shaped such that a force that is applied to one end of the slinky spring in a first direction may cause a force at the other end of the slinky spring in a second direction that is opposite to the first direction.

The invention further concerns an injector device comprising a needle ejection and retraction mechanism, a control unit which is adapted to control the actuator, and a sensor unit which is connected to the control unit and which is adapted to determine, after the needle has at least been partially ejected from the injector device, a value which indicates a distance between the sensor unit and the skin of a human or animal body, wherein the control unit is adapted, upon determination that the value exceeds a predetermined threshold value, to cause the actuator to activate the exertion of the force in the proximal direction on the needle hub so that a distal tip of the needle does not protrude from the injector device.

For example, upon determination of an abrupt loss of contact of the needle with the skin of the human or animal body, the control unit can cause the actuator to activate the exertion of the force in the proximal direction on the needle hub so that a distal tip of the needle does no longer protrude from the injector device. Thus, a safety feature is provided which allows an instantaneous retraction of the needle into the housing of the injector device once the injection of the needle into the skin is stopped, in particular, before the usual end of the injection process, or at the end of a normal injection process.

Preferably, all mechanical functions of the injector device may be carried out by the single actuator, i.e., opening of a door of the injector device in which the drug storage cartridge may be placed, locking and unlocking of the door, causing needle ejection and retraction, and pushing the drug out of the drug storage cartridge to dispense the drug via the needle into a human or animal body.

The invention further concerns an auto-injector device comprising a needle ejection and retraction mechanism.

Preferred embodiments of the invention will now be described in greater detail with reference to the appended schematic drawings, wherein:

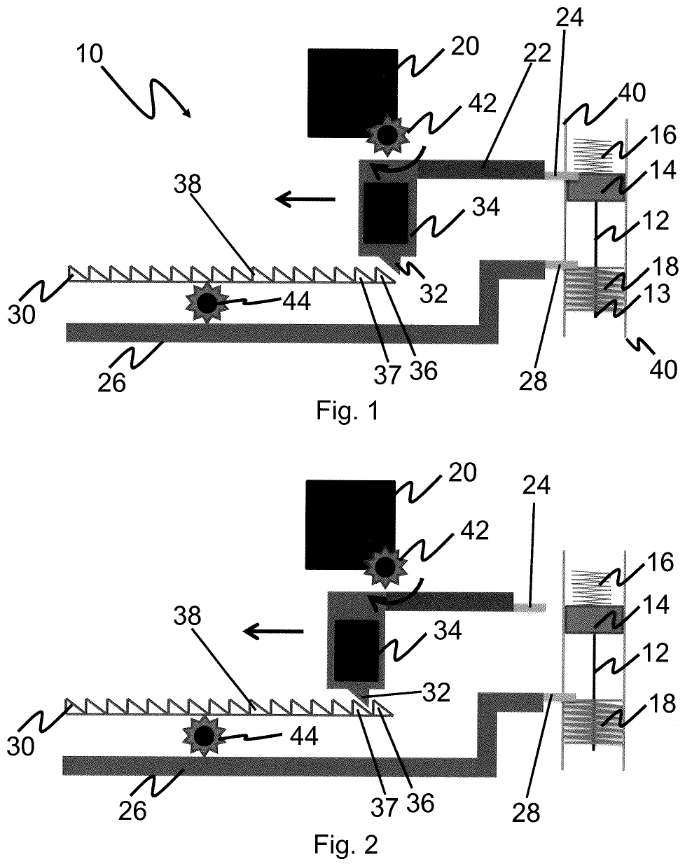

FIGS. 1 to 6 schematically show a first embodiment of a needle ejection and rejection mechanism;

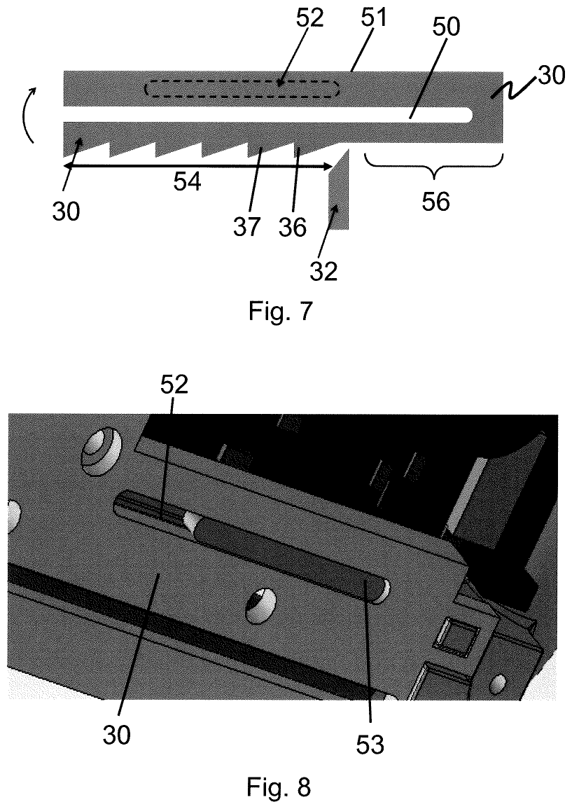

FIG. 7 schematically shows a ratchet and a pawl;

FIG. 8 shows a perspective view of a ratchet with a slider and a slot;

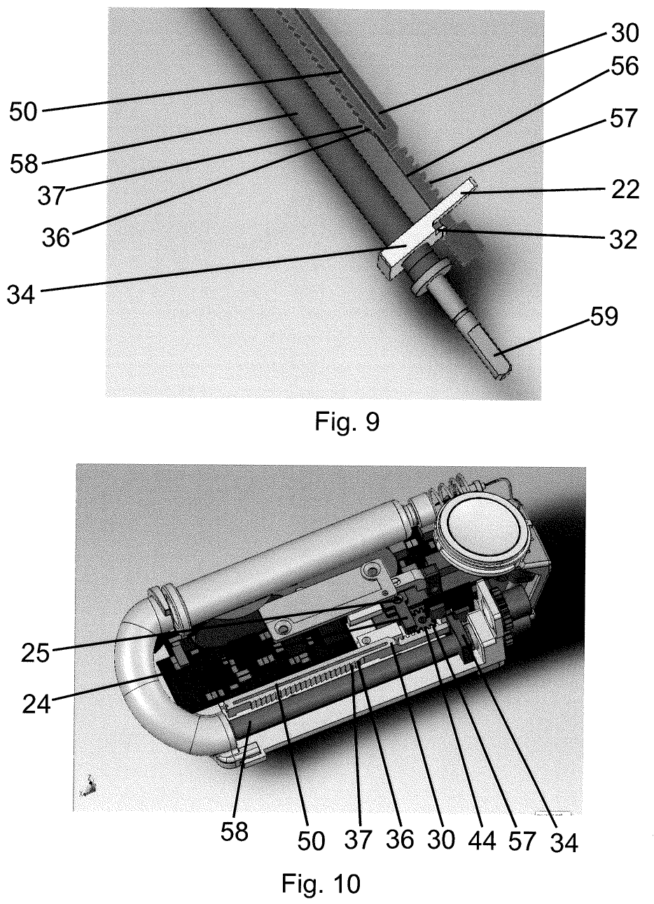

FIG. 9 shows a perspective view of a ratchet and a pawl;

FIG. 10 shows a perspective view of an injector device with a ratchet;

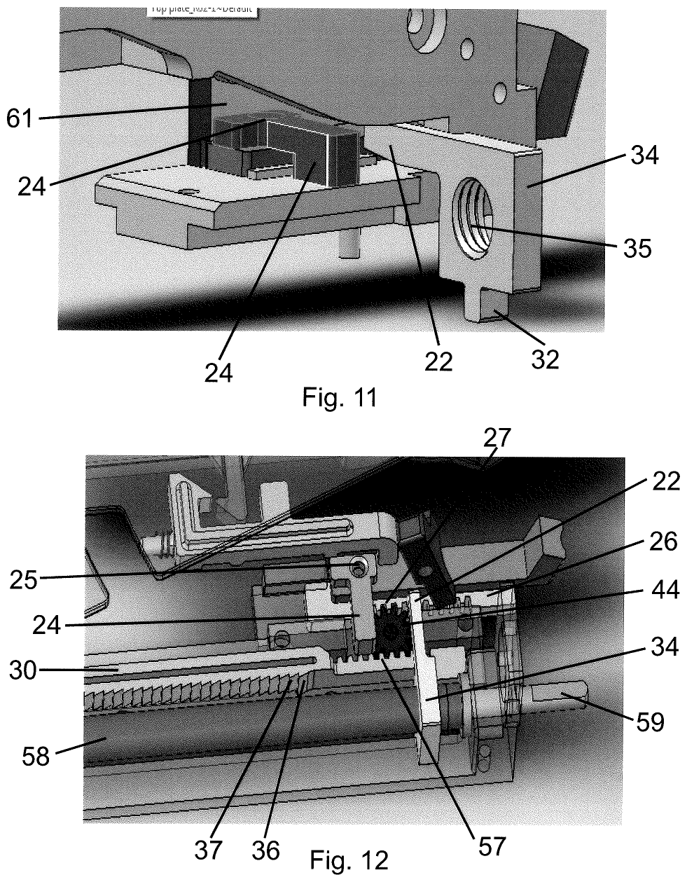

FIG. 11 shows a perspective view of a ratchet, a nut with a pawl, and a first lever;

FIG. 12 shows a perspective view of a ratchet, a nut with a pawl, and a first and a second lever;

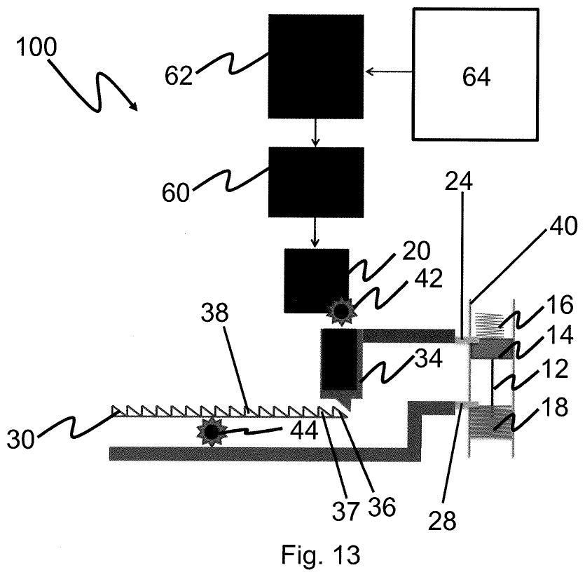

FIG. 13 schematically shows a second embodiment of a needle ejection and retraction mechanism with a skin sensor;

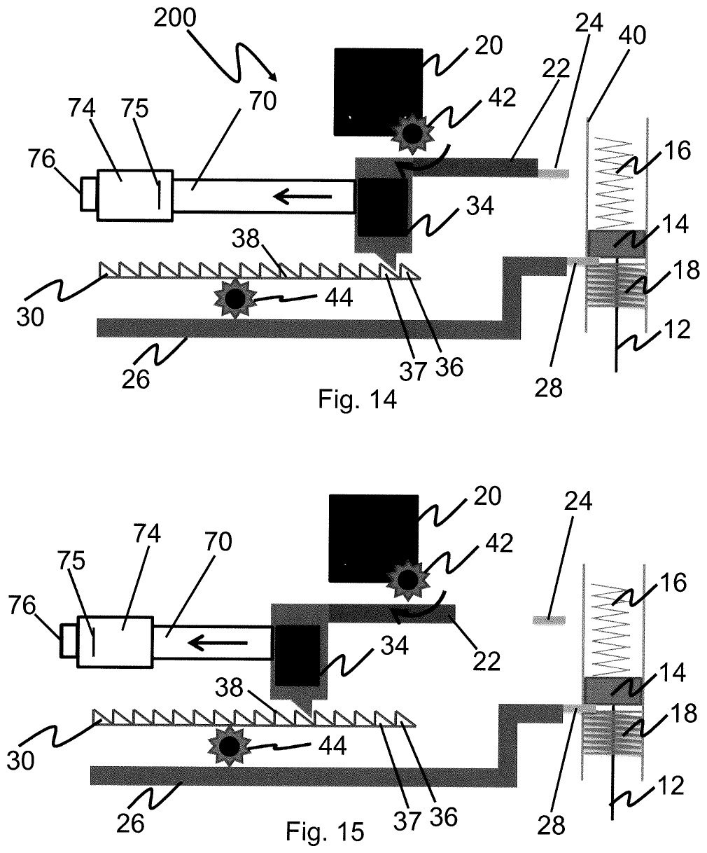

FIGS. 14 and 15 schematically show a third embodiment of a needle ejection and retraction mechanism with a push mechanism;

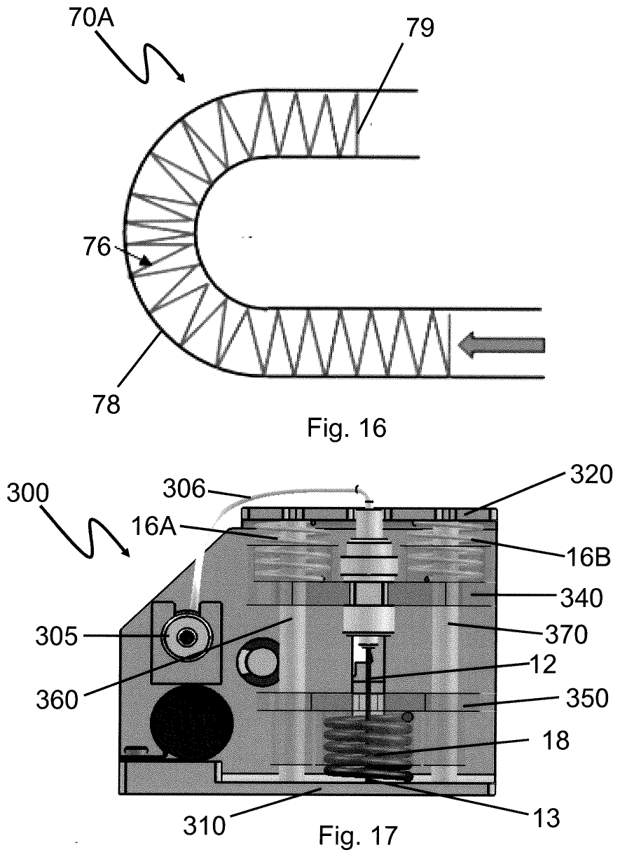

FIG. 16 schematically shows an embodiment of push mechanism; and

FIGS. 17 schematically shows a fourth embodiment of a needle ejection and retraction mechanism.

FIGS. 1 to 6 schematically show a needle ejection and retraction mechanism 10 according to a first embodiment. Specifically, FIGS. 1 to 6 show the operation of the needle ejection and retraction mechanism 10 during needle ejection and retraction.

As can be seen from FIG. 1, the needle ejection and retraction mechanism 10 comprises a needle 12 have at its distal end a needle tip 13 and at its proximal end a needle hub 14. Needle 12 is a hollow injection needle, and needle tip 13 is adapted to be injected to the skin of a human or animal body. Hollow needle 12 is configured such that an injectable drug may flow therethrough and may be expelled via the needle tip 13 into the skin of a human or animal body.

Needle hub 14 is extending perpendicularly from the proximal end of the needle 12. Needle 12 and needle hub 14 are adapted to be moved in the distal direction, i.e., towards the needle tip 13, and in the proximal direction, i.e., away from the needle tip 13. Needle 12 and needle hub 14 are provided such in a housing 40 that they are movable in the distal and in the proximal direction. For this, needle hub 14 may have, e.g., a round circumferential shape which fits to the inner shape of the housing 40. In FIGS. 1 to 6, housing 40 is only schematically illustrated as a channel. However, housing 40 may also be a housing that covers the entire needle ejection and retraction mechanism 10, for example, a housing of an auto-injector device or an injector device.

FIG. 1 shows an initial state of the needle ejection and retraction mechanism 10 in which the needle 12 is located inside the housing 40, i.e., the needle 12 and the needle tip 13 do not protrude from the housing 40 to the outside so that the needle tip 13 may not cause any injury.

The needle ejection and retraction mechanism 10 further comprises a first spring 16 and a second spring 18. The first 16 and second 18 springs are single coil springs. However, it is possible to replace each of the first 16 and second 18 springs by more than one spring and/or any other spring mechanism that is adapted to store a spring s force and selectively release the spring force. The first spring 16 is located proximally relative to the needle hub 14. In the initial state shown in FIG. 1, the first spring 16 is partially or fully compressed and adapted to release its spring force on a proximal side of the needle hub 14, i.e., a side that is facing the proximal direction, such that the spring force causes a movement of the needle hub 14 together with the needle 12 in the distal direction. Specifically, the first spring 16 is fixed at its proximal end to the housing 40 and at its distal end to the proximal side of the needle hub 14.

The second spring 18 is located distally relative to the needle hub 14 and the first spring 16. Moreover, the second spring 18 is located on the same longitudinal axis as the first spring 16, the needle hub 14, the needle 12 and the needle tip 13. In the initial state shown in FIG. 1, the second spring 18 is partially or fully compressed, is adapted to release its spring force in the proximal direction, and is provided such in the housing 40 that it does not contact the needle hub 14. Specifically, the second spring 18 is fixed at its distal end to the housing 40. The second spring 18 is further configured such that it can release a higher spring force than first spring 16.

In the initial state of the needle ejection and retraction mechanism 10 shown in FIG. 1, the first needle 16 and the second needle 18 are held in their compressed states. For this, the needle ejection and retraction mechanism 10 comprises a first spring force activation mechanism and a second spring force activation mechanism. The first spring force activation mechanism comprises a first lever 24, and the second spring force activation mechanism comprises a second lever 28 which is connected to a second lever arm 26. The first lever 24 holds the first spring 16 in its compressed state, and the second lever 28 holds the second spring 18 in its compressed state.

The needle ejection and retraction mechanism 10 further comprises a motor 20 and a ratchet mechanism. The ratchet mechanism comprises a ratchet 30 having a plurality of teeth 36, 37, 38 and a nut 34. The ratchet 30 has a linear shape and comprises a thread with the plurality of teeth 36, 37, 38, i.e., is formed as a toothed rack. At one end, the nut 34 comprises a pawl 32, and at the other end, the nut 34 comprises a first lever arm 22. The first lever arm 22 extends in a perpendicular from the nut 34. In particular, the first lever arm 22 extends in a direction that is perpendicular to the distal and proximal directions of the movement of the needle 12. The first lever arm 22 is configured to push the first lever 24 in a direction away from the first spring 16.

Moreover, the needle ejection and retraction mechanism 10 comprises a first gear 42 which is provided between the motor 20 and the nut 34, and a second gear 44 which is provided between the ratchet 30 and the second lever arm 26. A gear thread matching the first gear 42 is provided at at least one of the nut 34 and the first lever arm 22. The motor 20 is configured to cause a movement of the nut 34 in a direction away and a direction towards the first spring 16, in particular, a direction that is perpendicular to the distal end and proximal directions in which the needle 12 together with the needle hub 14 are moved. Specifically, as can be seen from FIGS. 1 and 2, the motor 20 causes a rotation of the first gear 42 in a clockwise direction, which causes a movement of the nut 34 together with the first lever arm 22 and the pawl 32 in the direction away from the first spring 16. By means of this movement, the first lever arm 22 pushes the first lever 24, thereby releasing the first spring 16..

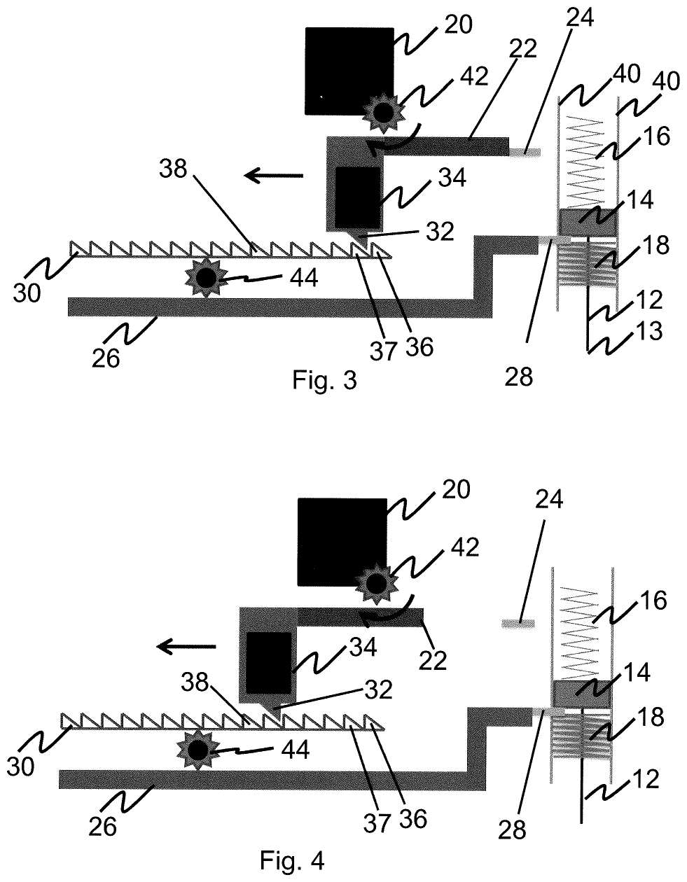

As can be seen from FIG. 3, the spring force that is exerted by the first spring 16 on the needle hub 14 causes a movement of the needle hub 14 together with the needle 12 in the distal direction such that a distal part of the needle 12 and the needle tip 13 are ejected out of the housing 40. In particular, the needle tip 13 is ejected such from the housing 40 that the needle tip 13 may be injected into a skin of a human or animal body (not shown in FIG. 3).

FIGS. 1 to 4 show the limited vertical space available in the needle ejection and retraction mechanism 10 and the arrangement of the springs 16 and 18 to achieve a vertical motion of the needle of about 7,33 mm, i.e., a vertical motion sufficient to produce a skin penetration depth of the needle 12 of about 6 mm.

The ejection of the needle 12 from the housing 40 may stop when the first spring 16 is completely released, when the needle hub 14 abuts the second lever 28, when the needle hub 14 abuts the second spring 18, or when then the needle hub 14 abuts a stopping member (not shown in FIG. 3). In the state shown in FIG. 3, the second spring 18 is still held by the second lever 28 in its compressed state.

When the motor 20 moves the nut 34 together with the first lever arm 22 and the pawl 32 in the direction away from the first spring 16, the ratchet 30 remains at a fixed location and the pawl 32 runs over the plurality of teeth 36, 37, 38. FIGS. 1 to 3 show how the pawl 32 runs over a first tooth 36 that is located closest to the first spring 16, and FIG. 4 shows how the pawl 32 runs over further teeth. This further movement of the nut 34 in the direction away from the first spring may cause an activation of a fluid dispense through the needle 12, which will be described in detail with regard to the third embodiment.

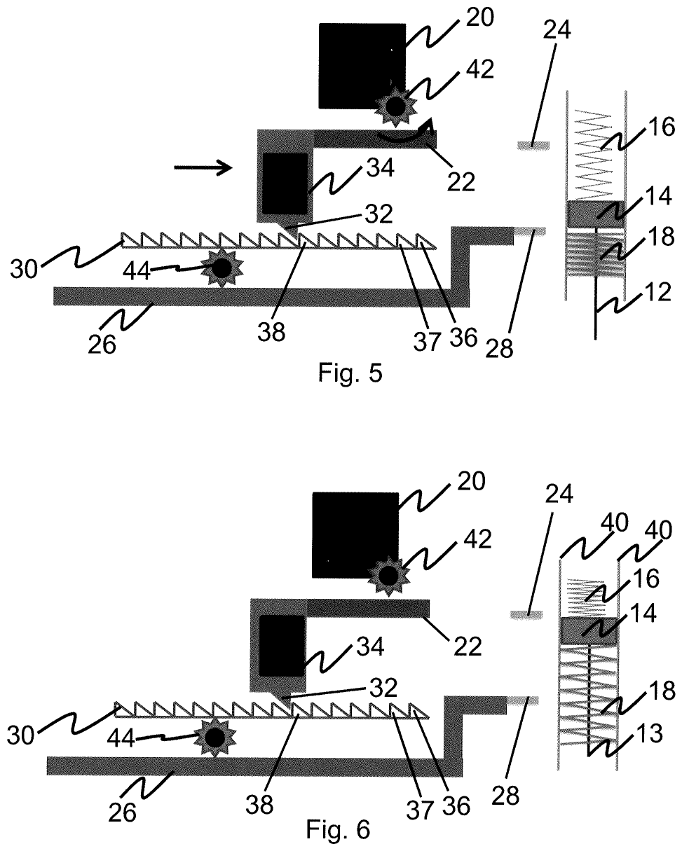

After the needle ejection process shown in FIGS. 1 to 4, the motor 20 causes a rotation of the first gear 42 in a counter clockwise direction, which causes a movement of the nut 34 together with the first lever arm 22 and the pawl 32 in a direction towards the first spring 16. This movement causes an engagement of the pawl 32 with one of the plurality of teeth 36, 37, 38, i.e., the first tooth 38 that is located next to the pawl 32 and closer to the first spring 16 than the pawl 32. As can be seen from FIG. 5, when the pawl 32 engages with the tooth 38 and the pawl 32 is further moved in the direction towards the first spring 16, the entire ratchet 30 is moved in the direction towards the first spring 16. By means of this movement of the ratchet 30 in the direction towards the first spring 16, the second gear 44 is rotated in a clockwise direction, which causes a movement of the second lever arm 26 and the second lever 28 in the direction away from the second spring 18. For this, a first gear thread may be provided at a side of the ratchet 30 that faces the lever arm 26 (not shown in FIG. 5), and a second gear thread may be provided at a side of the second lever arm 26 that faces the ratchet 30 (not shown in FIG. 5). Thus, the movement of the ratchet 30 in the direction towards the first spring 16 causes a displacement of the second lever 28 in a plane that is perpendicular to the direction in which the needle 12 is moved, thereby releasing the second spring 18 from its compressed state. In particular, as can be seen from FIG. 5, the second lever 28 is moved in a direction that is perpendicular to the direction in which the needle 12 is moved.

FIG. 6 shows how the second spring 18 releases its spring force on the needle hub 14, i.e., on a face of the needle hub 14 that faces the distal direction. Since the second spring 18 is adapted to release a higher spring force than the first spring 16, the second spring 18 pushes the needle hub 14 together with the needle 12 in the proximal direction such that the needle tip 13 is retracted into the housing 40 again, i.e., the needle tip 13 no longer protrudes from the housing 40 and may no longer cause any injury. Thereby, the first spring 16 is compressed again.

Since the ratchet 30 comprises a plurality of teeth 36, 37, 38, each of which may engage with the pawl 32 when the pawl 32 is moved in the direction towards the first spring 16, the spring force of the second spring 18 may at any time be released, thereby causing an immediate and swift retraction of the needle tip 13 into the housing 40. Thus, even in case the needle 12 is not completely ejected from the housing 40 in its final skin injection position or a drug delivery process through the hollow needle 12 is not completely finished or has not even started yet, the needle 12 may at any time be instantaneously retracted into the housing 40 such that the needle tip 13 no longer protrudes from the housing 40.

FIG. 7 schematically shows an embodiment of the ratchet 30 and the pawl 32. In FIG. 7, only a part of the ratchet 30 is illustrated, i.e., the ratchet 30 continues to the left and the right, as, for example, shown in FIG. 10. The ratchet 30 and the pawl 32 may be used in the first embodiment or any other embodiment described in this disclosure. In FIG. 7, compared to FIGS. 1 to 6, the ratchet 30 is shown in a view that is rotated by 180 degrees and shown from the other side. Ratchet 30 comprises an opening 50 which extends along the plurality of teeth 36, 37. Opening 50 is formed like a window and has a slot-shape. Ratchet 30 is made of a plastic material and comprises a surface 51 which faces away from the plurality of teeth 36, 37. The opening 50 extends along the plurality of teeth 36, 37 and is provided between the plurality of teeth 36, 37 and the surface 51 that faces away from the plurality of teeth 36, 37. Thus, when the pawl 32 is moved over the plurality of teeth 36, 37 in the direction away from the first spring 16 (not shown in FIG. 7), the part 54 of the ratchet 30 comprising the plurality of teeth 36, 37 flexes towards the surface 51 which faces away from the plurality of teeth 36, 37 so that the pawl 32 can slide over the edges of the teeth 36, 37. In other words, the pawl 32 is only moved in the direction along the plurality of teeth 36, 37, however, not in a direction towards and away from plurality of teeth 36, 37.

The surface of the ratchet 30 which is facing the pawl 32 comprises a first area 54 and a second area 56. The second area 56 is located closer to the first 16 and second springs 18 than the first area 54 (not shown in FIG. 7). The first area 54 comprises the plurality of teeth 36, 37, and the second area comprises no teeth. The pawl 32 is adapted to be moved over the second area 56 and the first area 54. When the pawl 32 is located at the second area 56 and is moved towards the first area 54, it runs over the plurality of teeth 36, 37. Thereafter, when the pawl 32 is moved towards the second area 56, it engages with one of the plurality of teeth 36 and moves the ratchet 30. Since no teeth are provided in the second area 56, it is ensured that the pawl 32 can slide over the edge of the first tooth 36 closest to the first 16 and second springs 18, i.e., also the first tooth 36 is pushed by the pawl 32 towards the surface 51 which faces away from the plurality of teeth 36, 37 (not shown in FIG. 7).

For guiding the movement of the ratchet 30, the ratchet 30 comprises a slot 52, which is located at a part of the ratchet 30 that is opposite the plurality of teeth 36, 37. A slider fixed to a base plate is provided in the slot 52, and the slot 52 guides the movement of the ratchet 30 towards the first 16 and second springs 18 (not shown in FIG. 7). FIG. 8 shows a perspective view of the ratchet 30 of FIG. 7 with the slot 52 and a slider 53.

FIG. 9 shows a perspective view of the ratchet 30 of FIGS. 7 and 8 together with the nut 34, the pawl 32, and the first lever arm 22. In FIG. 9, only one part of the ratchet 30 is illustrated, i.e., the ratchet 30 continues to the upper left, as, for example, shown in FIG. 10. The nut 34 can be moved along the ratchet 30 by means of leadscrew 58 which is provided in parallel to the extension direction of the ratchet 30. At the end of the leadscrew 58 which is close to the first 16 and second 18 springs (not shown in FIG. 9), the leadscrew 58 comprises a driving gear 59 that can be driven, i.e., rotated, by the motor 20 (not shown in FIG. 9).

FIG. 10 shows a perspective view of an injector device with the ratchet 30. In FIG. 10, the entire ratchet 30 with the opening 50 is illustrated.

As can be seen from FIG. 11, the nut 34 comprises a female inner thread 35 which fits the leadscrew 58 as a male thread so that a rotation of the driving gear 59 by the motor 20 causes a movement of the nut 34, the pawl 32, and the first lever arm 22 along the leadscrew 58. FIGS. 9, 10 and 12 further show that the ratchet 30 comprises a thread 57 which is located opposite the second area 56 and is adapted to be engaged with a gear 44 (not shown in FIG. 9).

FIGS. 10 to 12 show how the first lever arm 22 pushes the first lever 24. The nut 34 comprising the first lever arm 22 and the pawl 32 is substantially L-shaped and comprises in the middle between the first lever arm 22 and the pawl 32 the female inner thread 35. In particular, the first lever 24 extends in a direction that is perpendicular to the direction in which the pawl 32 extends. The first lever 24 is substantially L-shaped and comprises a pin 25. Initially, when the first lever arm 22 pushes the first lever 24, the first lever 24 is guided by a wall 61 and is moved linearly along the wall 61, thereby releasing the first spring 16 (not shown in FIGS. 10 to 12). At the end of the wall 61, the first lever 24 is rotated around the pin 25 by 90 degrees so that the first lever 24 is not obstructing the first lever arm 22 from being moved backwards in the opposite direction.

FIG. 12 further shows that the second lever arm 26 comprises a thread 27 and how a movement of the ratchet 30 with the thread 57 causes a rotation of the second gear 44, which causes a movement of the second lever arm 26 such that the second spring 18 (not shown in FIG. 12) is released.

FIG. 13 shows a second embodiment of a needle ejection and retraction mechanism 100. The second embodiment is based on the first embodiment. Thus, the same reference signs of the second embodiment relate to the same elements of the first embodiment, and any repetition of these elements is omitted.

In addition to the first embodiment according to FIGS. 1 and 6, the second embodiment according to FIG. 13 comprises a motor control unit 60, a system control unit 62, and a sensor unit 64.

The motor 20 is a single direct current (DC) electric motor that is controlled by the motor control unit 60 in order to cause the ejection and subsequent retraction of the needle 12. The motor 20 is adapted to provide a relatively high torque. The speed of the motor 20 is controlled by the motor control unit 60 via a pulse-width modulation (PWM) control scheme.

The sensor unit 64 is adapted to determine, after the needle 12 has at least been partially ejected from the injector device 100, a value which indicates a distance between the sensor unit 64 and the skin of a human or animal body. The motor control unit 60 is adapted, upon determination that the value exceeds a predetermined threshold value, to cause the motor 20 to activate the exertion of the force in the proximal direction on the needle hub 14 so that a distal tip of the needle 12 does no longer protrude from the injector device 100. For example, the sensor unit 64 may determine when at least one of the needle 12 and the housing 40 is no longer in contact with the human or animal body or the needle 12 has been abruptly removed from the skin. The sensor unit 64 may comprise a skin sensor array. Particularly, the sensor unit 64 may comprise a proximity sensor using a capacitive sensing technology which can determine a distance between the proximity sensor and the skin of the human or animal body.

After the system controller 62 has received a signal from the sensor unit 64 which indicates that the determined value exceeds a predetermined threshold value, the system controller 62 instructs the motor control unit 60 to provide respective motor drive signals to the motor 20. In particular, when applying the second embodiment according to FIG. 13 on FIGS. 5 and 6 of the first embodiment, upon detection by the sensor unit 64 that the determined value exceeds a predetermined threshold value, the system control unit 62 causes, via the motor control unit 60, the motor 20 to drive the first gear 42 in the counter clockwise direction, which causes a movement of the ratchet 30 in the direction towards the first 16 and second 18 springs, thereby causing a release of the second spring 18 and an immediate retraction of the needle 12 into the housing 40, as can be seen from FIG. 6.

FIGS. 14 and 15 schematically show a third embodiment of a needle ejection and retraction mechanism 200. The third embodiment is based on the first and second is embodiments. Thus, the same reference signs of the third embodiment relate to the same elements of the first and second embodiments, and any repetition of these elements is omitted.

FIGS. 14 and 15 correspond to FIGS. 3 and 4 of the first embodiment. However, in addition to the first embodiment, FIGS. 14 and 15 schematically show a push mechanism 70 and a drug storage cartridge 74. The drug storage cartridge 74 is adapted to store an injectable drug and comprises a movable piston 75 and an opening 76. Moving the piston 75 towards the opening 76 forces the drug out of the opening 76. The opening 76 is connected to the proximal end of the needle 12 such that the drug may be dispensed via the hollow needle 12 and the needle tip 13 into a human or animal body (not shown in FIGS. 14 and 15).

After the first lever 24 has caused the ejection of the needle 12, as shown in FIG. 13, the motor 20 causes a further movement of the nut 34 in the direction away from the first spring 16. Thereby, the nut 34 pushes the push mechanism 70 such that it causes a movement of the piston 75. Thus, the drug which is stored in the drug storage cartridge 74 is forced via the opening and the needle 12 out of the tip of the needle 13.

Accordingly, the movement of the nut 34 together with the first lever 24 and the pawl 32 caused by the single motor 20 in the direction away from the first spring 16 has a double function of releasing the first spring 16 and thereafter forcing the drug out of the drug storage cartridge 74.

FIG. 16 schematically shows an embodiment of a push mechanism 70A. The push mechanism 70A may be used as the push mechanism 70 in the third embodiment.

The push mechanism 70A comprises a spring guide 78 and slinky spring 76 which is adapted to be moved in the spring guide 78. The slinky spring 76 may be made of a metal material. The spring guide 78 has a curved shape, for example a U-shape. At a proximal end, the slinky spring 76 is adapted to be pushed by the nut 34, as indicated by the arrow. At a distal end, the slinky spring 76 comprises a cap 79 that is adapted to push the piston 75 of the drug storage cartridge 74 shown in FIGS. 14 and 15. Thus, the push mechanism 70A enables a turning of the direction of the pushing force by 90 degrees. Hence, it is possible to include the needle ejection and retraction mechanism in a small-sized injector device, for example, an injector device having a shape like a computer mouse with a flat surface that is adapted to be placed on a human or animal body and through which the needle 12 is ejected.

FIG. 17 schematically shows a fourth embodiment of a needle ejection and retraction mechanism 300. The same reference signs of the fourth embodiment relate to the same elements of the first to third embodiments, and any repetition of these elements is omitted.

The needle ejection and retraction mechanism 300 is provided in a housing of an injector have a base plate 310 and a top plate 320. Between the base plate 310 and the top plate 320, two vertical rods 360 and 370 are foreseen. The outer surface of the base plate 310 is adapted to be placed on the skin of the human or animal body. Upon ejection of the needle 12, the needle tip 13 protrudes from the outer surface of the base plate 310 (not shown in FIG. 17).

The fourth embodiment differs from the first embodiment in that the first spring 16 is replaced by two first springs 16A and 16B. The two first springs 16A and 16B extend in parallel to an axis along which the needle 12 is ejected and are provided at a same distance from this axis. The two first springs 16A and 16B are provided between the top plate 320 and a first support element 340 which is adapted to release the spring force by the two first springs 16A and 16B on the needle 12. The second spring 18 is provided between the base plate 310 and a second support element 350. FIG. 17 further shows a drug storage cartridge 305 which is connected via a connecting tube 306 to the proximal end of the hollow needle 12.

1. A needle ejection and retraction mechanism-00, comprising:
a needle adapted to be injected into the skin of a human or animal body and through which an injectable drug may flow;
a needle hub connected to the needle and adapted to be displaced in a distal direction and in a proximal direction;
a first spring mechanism compressed to exert a force in the distal direction on the needle hub and adapted to cause the needle to eject in the distal direction;
a second spring mechanism compressed to exert a force in the proximal direction on the needle hub and adapted to cause the needle to retract in the proximal direction after it has been ejected in the distal direction; and
an actuator which is adapted to activate the exertion of the force in the distal direction on the needle hub and the exertion of the force in the proximal direction on the needle hub,
wherein the second spring mechanism is adapted to cause a compression of the first mechanism spring after the needle has been ejected in the distal direction and before a drug delivery process through the needle is completely finished.

2. The needle ejection and retraction mechanism 00 according to claim 1, wherein
the second spring mechanism is adapted to exert a higher force than the first mechanism spring.

3. The needle ejection and retraction mechanism 00 according to claim 1, further comprising
a first spring force activation mechanism which is adapted to hold the first spring mechanism in the compressed state and to release the first spring mechanism from the compressed state in order to exert the force in the distal direction on the needle hub; and
a second spring force activation mechanism which is adapted to hold the second spring mechanism in the compressed state and to release the second spring mechanism from the compressed state in order to exert the force in the proximal direction on the needle hub.

4. The needle ejection and retraction mechanism according to claim 3, wherein
the first spring force activation mechanism comprises a first lever adapted to hold the first spring mechanism in the compressed state; and
the second spring force activation mechanism comprises a second lever which is adapted to hold the second spring mechanism in the compressed state, wherein
at least one of the first lever and the second lever is adapted to be moved in a direction that differs from the distal direction and the proximal direction.

5. The needle ejection and retraction mechanism according to claim 4, wherein the first lever is adapted to be rotated around an axis that is substantially parallel to the distal direction and the proximal direction after it has been moved in a direction that differs from the distal direction and the proximal direction.

6. The needle ejection and retraction mechanism according to claim 1, further comprising:
a ratchet mechanism which is coupled to the actuator, the first spring force activation mechanism, and the second spring force activation mechanism, wherein the ratchet mechanism is adapted to activate the exertion of the force in the distal direction on the needle hub and thereafter activate the exertion of the force in the proximal direction on the needle hub, wherein the ratchet mechanism comprises a ratchet and a pawl,
wherein:
the ratchet comprises a plurality of teeth,
the pawl is adapted to engage with the teeth,
the pawl is coupled to the first spring force activation mechanism, and
the ratchet is coupled to the second spring force activation mechanism.

7. The needle ejection and retraction mechanism according to claim 6, wherein the ratchet comprises a surface which faces away from the plurality of teeth and an opening which extends along the plurality of teeth and is provided between the plurality of teeth and the surface that faces away from the plurality of teeth.

8. The needle ejection and retraction mechanism according to claim 7, wherein:
a surface of the ratchet facing the pawl comprises a first area and a second area, wherein the second area is located closer to the first and second spring mechanisms than the first area, the first area comprises the plurality of teeth, the second area comprises no teeth and extends along the opening, and the pawl is adapted to be moved along the second area and the first area.

9. The needle ejection and retraction mechanism according to claim 6, wherein the actuator is adapted to move the pawl in a first direction away from the first spring mechanism and thereby activate the exertion of the force in the distal direction on the needle hub.

10. The needle ejection and retraction mechanism according to claim 6, wherein the actuator is adapted to move the pawl in a second direction towards the first spring mechanism and thereby activate the exertion of the force in the proximal direction on the needle hub.

11. The needle ejection and retraction mechanism according to claim 10, wherein the pawl is adapted, when being engaged with one of the plurality of teeth, to move the ratchet in the second direction and thereby activate the exertion of the force in the proximal direction on the needle hub.

12. The needle ejection and retraction mechanism according to any one of claim 6, further comprising
a push mechanism which is coupled to the pawl, wherein the push mechanism is adapted, upon movement of the pawl over the plurality of teeth, to initiate a process of forcing a drug out of a drug storage cartridge.

13. The needle ejection and retraction mechanism according to claim 12, wherein
the push mechanism comprises a spring guide and a slinky spring which is adapted to be moved in the spring guide, wherein the spring guide has a curved shape.

14. An injector device, comprising the needle ejection and retraction mechanism according to claim 1;
a control unit is adapted to control the actuator; and
a sensor unit is connected to the control unit and adapted to determine, after the needle has at least been partially ejected from the injector device, a value indicative of a distance between the sensor unit and the skin of a human or animal body, wherein
the control unit is adapted, upon determination that the value exceeds a predetermined threshold value, to cause the actuator to activate the exertion of the force in the proximal direction on the needle hub so that a distal tip of the needle does not protrude from the injector device.
